(19) United States
(12) Patent Application Publication
(10) Pub. No.: US 2007/0054953 A1
(43) Pub. Date: Mar. 8, 2007

(54) A NOVEL PROCESS FOR PREPARATION OF INDOLE DERIVATIVES

(76) Inventor: Ramesh Babu Potluri, (US)

Correspondence Address: Ramesh Babu Vishwanath;Vishwanath & Company, #48 Srt, 1 st Floor, Prakash Nagar, Begumpet,, Hyderabad, 500 016, IN

(21) Appl. No.: 10/556,345

(22) PCT Filed: May 12, 2003
(86) PCT No.: PCT/IN03/00183

§ 371 Date: Nov. 10, 2005

(51) Int. Cl. A61K 31/405; C07D 209/18

(52) U.S. Cl. 514/414; 514/419; 548/465; 548/495

(57) ABSTRACT

A novel process of preparation of a compound of 3-(2-Dimethylamino)-N-methyl-1H-indole-5-methane sulfonamide, which comprises of a reaction 4-hydrazino-N-methyl benzene methane sulfonamide with 4-dimethyl amino butyraldehyde diethyl acetal in a chlorinated solvent in the presence of ethyl poly phosphate and conversion of the crude product to a product of 3-(2-Dimethylamino)-N-methyl-1H-indole-5-methane sulfonamide succinate of extra high purity and colour.

### FIELD OF INVENTION

The present invention relates to a novel process for the preparation of Indole derivatives and to novel intermediates and more particularly to a process for the preparation of 3-(2-Dimethylamino)-N-methyl-1H-indole-5-methane sulfonamide succinate and pharmacologically acceptable salt of high purity.

3-(2-Dimethylamino)-N-methyl-1H-indole-5-methane sulfonamide succinate, which is represented by the formula IV

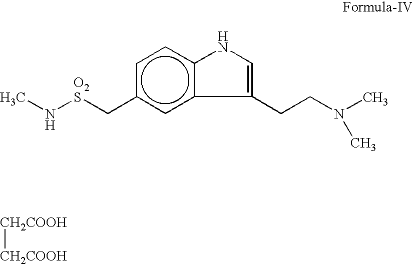

### BACKGROUND OF INVENTION

Indole derivatives of the following formula are known as pharmaceutical active ingredients or are important precursors in the preparation thereof. An important indole derivative is fluvastatin, an HMG-CoA reductase inhibitor, that is to say an inhibitor of biosynthesis of cholesterol, which is used in the treatment of hyperlipoproteinamia and arteriosclerosis.

3-(2-Dimethylamino)-N-methyl-1H-indole-5-methane sulfonamide is the pharmacologically acceptable salt, which exhibits selective vasoconstrictor activity and is indicated for use in the treatment of migraine.

In the preparation of 3-(2-Dimethylamino)-N-methyl-1H-indole-5-methane sulfonamide there exist different processes. Most of the processes prepare the 3-(2-Dimethylamino)-N-methyl-1H-indole-5-methane sulfonamide through reductive methylation which furnishes the N,N dimethyl derivative viz., sumatriptan. The reductive methylation step-leads to, a number of impurities and the purification is very difficult.

Some of the other methods make use of N,N-dimethyl amino butyraldehyde dimethyl acetal. Both N,N-dimethyl amino butyraldehyde dimethyl acetal as well as diethyl acetal are sysnthesised. The preparation of dimethyl acetal did not yield product of good quality and the yield was also less.

The inventor with his novel process of preparation of 3-(2-Dimethylamino)-N-methyl-1H-indole-5-methane sulfonamide, has explained to obtain the succinate salt of high purity and better colour.

### RELATED PRIOR ART

Here it appears to be relevant in discussing about the related applications that are already filed by the different inventors.

In DE3444572 describes the process as given below. 4-Hydrozino-N-methyl benzene methane sulfonamide is reacted with 4-chloro butyraldehyde dimethyl acetal to yield compound of formula VI. This compound was converted into compound of formula I by reductive methylation.

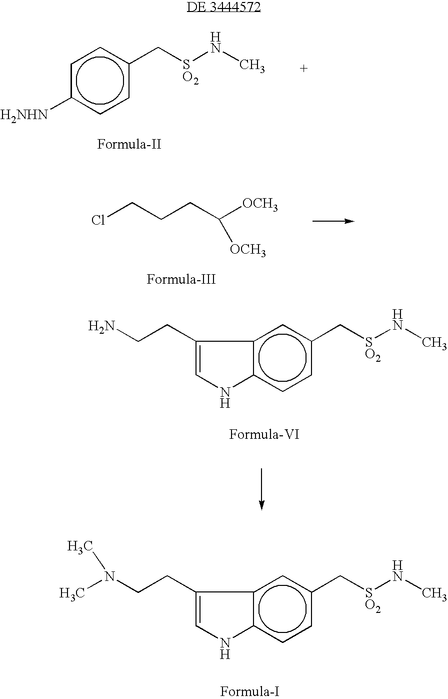

The process described in patent specification No. DE3700407 relates to the use of 4-(4,4-dimethyloxy butyl)phthalimide. Here compound of formula II is reacted with N-(4,4-dimethyloxy butyl)-phthalimide of formula VII to give a compound of formula VII, which is deprotected and reductively methylated to yield compound of formula I.

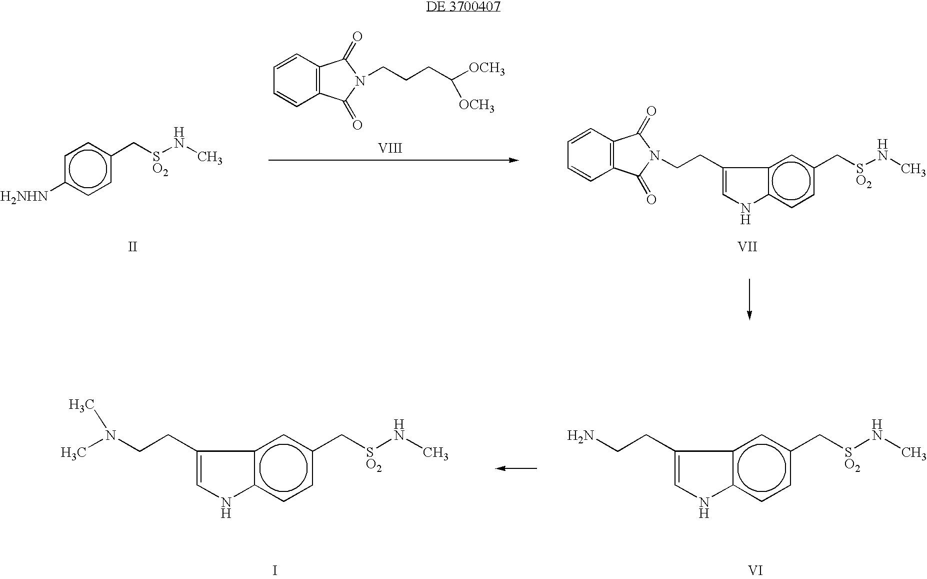

In the patent specification No. DE3700408, the use of N—N-dimethyl amino butyraldehyde dimethyl acetal is described. Compound of formula II is condensed with compound of formula III to give the hydrozone of formula IX, which was converted to compound of formula I.

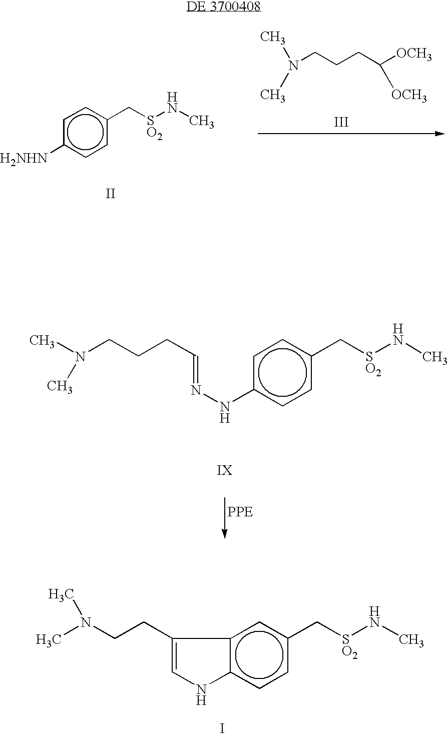

The patent specification No. EP145459 is as described. Phenyl-2-(4-nitrophenyl)-methane sulphonate of formula X is hydrogenated to the compound of formula XI, which was converted to the compound of formula XII. Condensation of the compound of formula XII with 4,4-dimethyoxy butyl cyanide of formula IIIa followed by Fischer indole cyclisation gave the indole derivative of formula XIII. Reduction of the cyano group gave the 3-(2-aminoethyl) derivative of indole of formula XIV, which was subjected to reductive methylation followed by reaction with monomehtylamine to give compound of formula I.

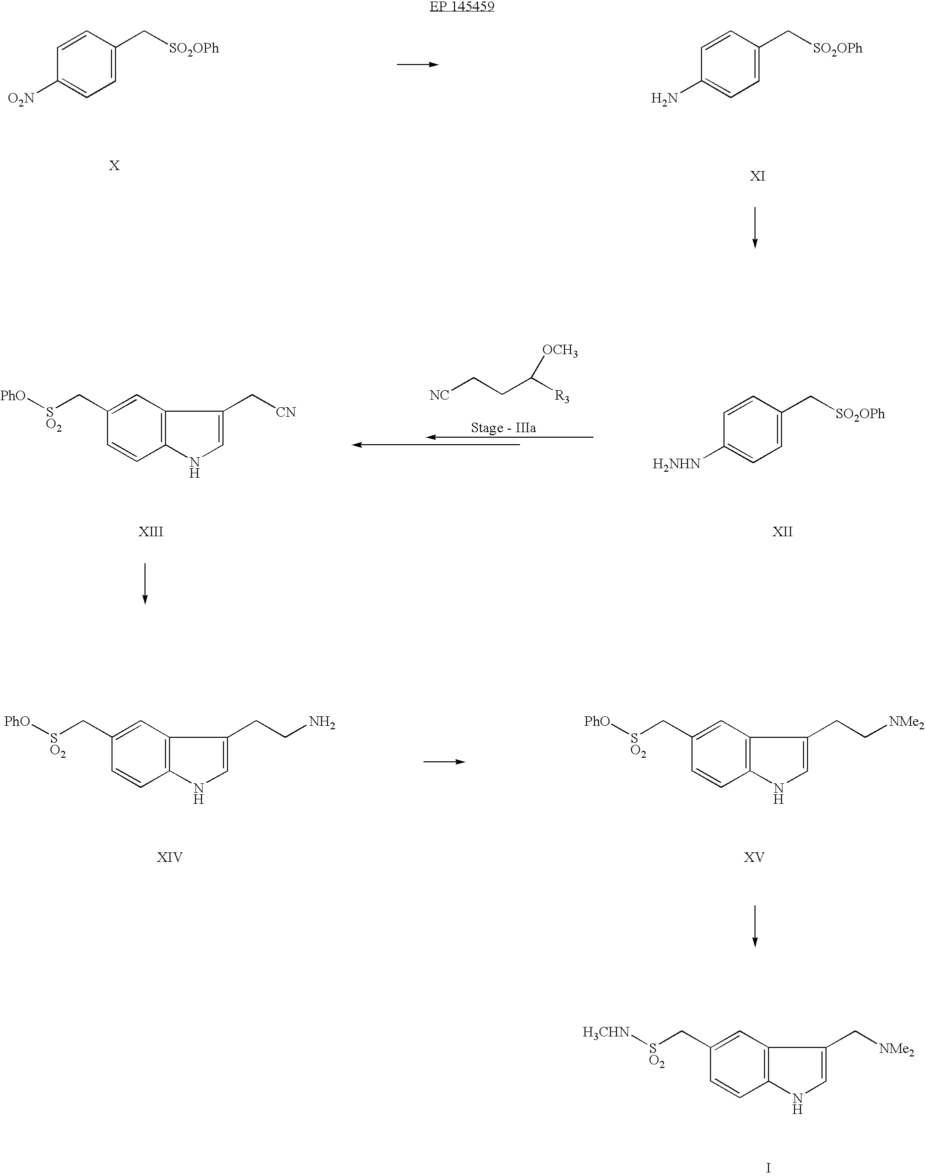

In the publication of JOC18,1356 (1953) both. N,N-dimethyl amino butyraldehyde dimethyl acetal as well as diethyl acetal are synthesized.

The process of dimethyl acetal did not yield-product of good quality and the yield was also less. Hence it was decided to concentrate on the preparation of diethyl acetal of formula III. The JOC 18,1356 (1953) describes the reaction of Grignard reagent with triethyl ortho formate in boiling benzene. It was observed that the reaction could be carried out in aromatic hydrocarbons like toluene, xylene or aliphatic hydrocarbons like hexane or alicyclic hydrocarbons like cyclopeptane, cyclohexane etc.

Since it was necessary to eliminate a carcinogenic solvent like benzene, the reaction was studied in the above mentioned solvents and cyclohexane was found to be solvent acceptable in terms of yield and purity of N,N-dimethyl amino butyraldehyde diethyl acetal.

The formation of hydrazone was carried out in hydrochloric acid. Reaction of the compound of formula II with compound of formula III to give a compound of formula IX was studied in dilute hydrochloric acid, hydrochloric acid of strength of 1N to 6N is found to be suitable. It is more preferable if the acid strength is about 1.5N to 4.5N. It is most preferable if the acid strength is about 2N to 3N. Conversion of the compound of formula IX to I was studied with reagents like dilute sulphuric acid, zinc chloride, acetic acid, ethyl polyphosphate etc. It was observed that ethyl polyphosphate was most suitable in terms of quality and yield of the product of formula I.

The weight ratio of the compound of formula II to that of ethyl polyphosphate is preferred to be 1:15. A weight ratio of 1:10 is more preferred. A weight ratio of 1:7 is most preferred. Dialkyl ethers, chlorocarbons etc., are suitable for the cyclisation reaction. The chloro carbons are more suitable for the reaction. The solvent, which is most suitable, is found to be chloroform. A reaction temperature of 15 to 65° C. is preferred. A reaction temperature of 25-45° C. is more preferred. A reaction temperature of 25 to 35° C. is most preferred.

After the reaction, the product is extracted into water. The base i.e., the compound of formula I is released by using an organic base like alkali/alkaline earth hydroxide, alkali/alkaline earth carbonates/bicarbonates, ammonia etc. it was preferred to use alkali/alkali earth carbonate/bicarbonate. It is most preferable to use alkali carbonates e.g. sodium/potassium carbonate. The released base is extracted with a suitable organic solvent and after washing; the solvent is stripped off under reduced pressure. The crude base is crystallized from a suitable solvent like aliphatic ketone, aliphatic nitriles, aliphatic carboxylic acid esters etc. The preferred solvents are aliphatic ketones or aliphatic nitrites. The most preferred solvents are aliphatic nitrile e.g. acetonitrile. The crude base has HPLC purity of about 80%. The crude base is further purified by recrystallisation. The preferred solvents are aliphatic nitrites, aliphatic ketones, dialkyl ethers, aliphatic carboxylic acid esters etc. The more preferred solvents are aliphatic ketones like acetone, methyl ethyl ketone etc. The recrystallised base has HPLC purity of about 98.0-98.5%. The conversion of recrystallised base of formula I to succinate of formula IV yield a product with HPLC purity of 99.1%-99.3%. The colour of the product is formed to be pale yellow.

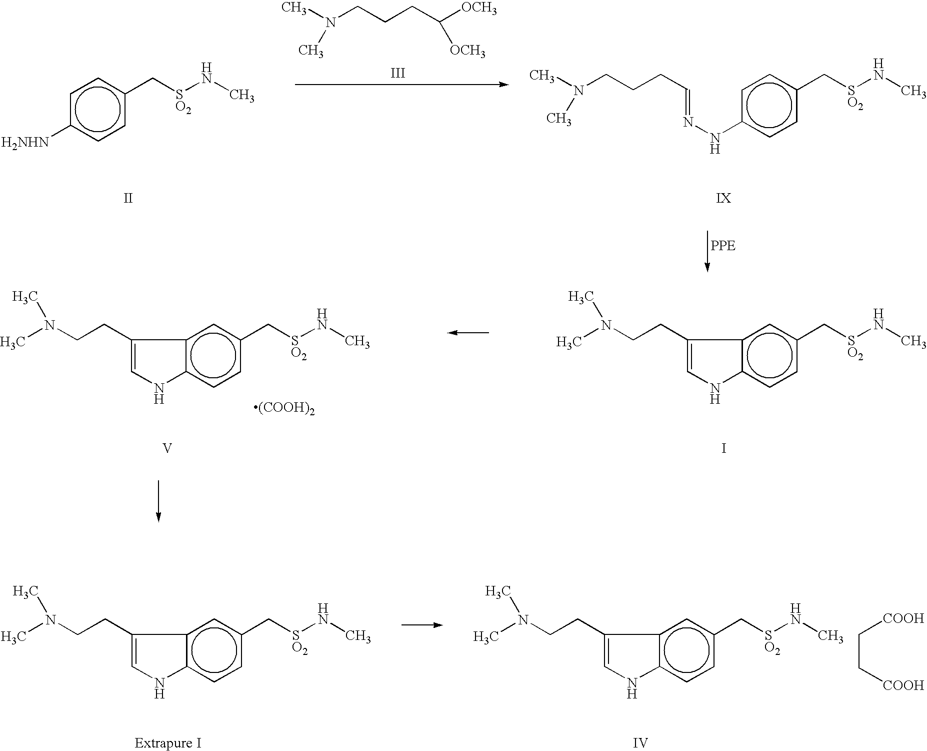

### SUMMARY OF THE INVENTION

A high purity and better colour of the succinate salt of 3-(2-Dimethylamino)-N-methyl-1H-indole-5-methane sulfonamide is obtained by the reaction of 4-hydrozino-N-methyl benzene methane sulfonamide (II) with 4-dimethyl amino butyraldehyde diethyl acetal (III) in a chlorinated solvent in the presence of ethyl poly phosphate and conversion of the crude product to a product of formula IV.

A method for the preparation of 3-(2-Dimethylamino)-N-methyl-1H-indole-5-methane sulfonamide (I) 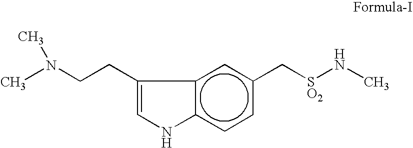
by the reaction of 4-hydrazino-N-methyl benzene methane sulfonamide (II) 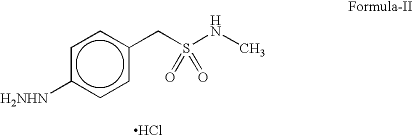
with 4-dimethyl amino butyraldehyde diethyl acetal (III) 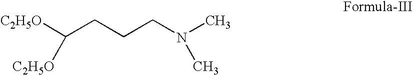
in a chlorinated solvent in the presence of ethyl poly phosphate and conversion of the crude product to a product of formula IV of extra high purity is described.

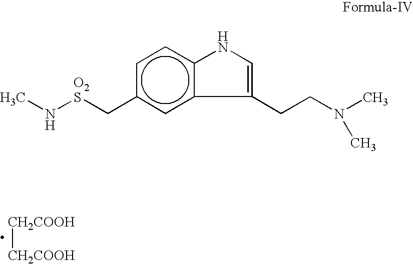

### DESCRIPTION OF THE INVENTION

This invention discloses the process for the preparation of 3-(2-Dimethylamino)-N-methyl-1H-indole-5-methane sulfonamide and pharmacological acceptable salt of high purity. 3-(2-Dimethylamino)-N-methyl-1H-indole-5-methane sulfonamide, which is represented by the formula I,

3-(2-Dimethylamino)-N-methyl-1H-indole-5-methane sulfonamide, which is represented by the formula I 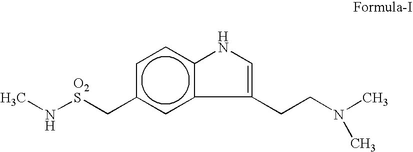
and pharmacologically acceptable salt viz., represented by the formula IV 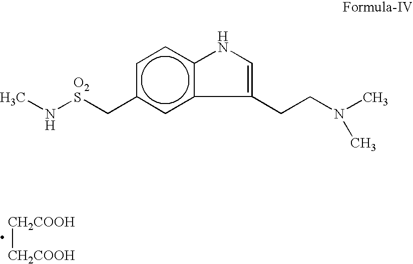
exhibits selective vasoconstrictor activity and are indicated for use in the treatment of migraine.

The present process describes the methodology to get a base and subsequently succinate salt of very excellent purity as well as colour. The recrystallised base with HPLC purity of 98.5%, as obtained through the process described in the prior art, is converted into a citrate/ascorbate/oxalate. The mole ratio of the carboxylic acid to the compound of formula I is preferred to be 1:5. It is more preferred to have a mole ratio of 1:3. It is most preferred to be maintain a mole ratio of 1:1.5 ñ2.0. The salt formation can be carried out in solvents like pure alkanols, alkanols containing water, alkoxy alkanols etc. Alkanol or alkanol containing water are more preferable. The salt formation takes place at temperature range of 15° C. to 100° C. The preferred temperature range for the salt formation is 25° C. to 85° C. A temperature range of 40-60° C. is most preferable. The salt is washed with the same solvent, which is used for the salt formation and the isolated salt is dried. The salt is dissolved in water and the base of formula I is precipitated by neutralizing with inorganic base like alkali/alkaline earth hydroxide, alkali/alkaline earth carbonates like sodium/potassium carbonates.

The precipitated base is filtered, washed with water (0-5° C.) and dried. The dried base can be further purified by recrystallisation using aliphatic ketones like acetones or methyl ethyl ketone.

The recrystallised base formula I has an HPLC purity of 99.4% to 99.6%. The succinate salt obtained from this base has an HPLC purity of 99.7% to 99.8% having off white to white colour. The yield obtained for similar batch size is similar for both the methodologies. This establishes the superiority of the present invention.

### ADVANTAGES OF THE INVENTION

The invention disclosure has advantage of the recrystallised base formula I has an HPLC purity of 99.4% to 99.6%. The succinate salt, obtained from this base has an HPLC purity of 99.7% to 99.8% having off white-to-white colour, which is not obtained in the earlier disclosures.

### EXAMPLES

The invention is further illustrated by the following examples, which should not be construed as limiting the scope of the invention. The melting points are uncorrected and the high pressure liquid chromatography has been conducted on Shimazu system with C-18 column (Normal and Reverse phase).

### Example 1

### 4,4-Dimethyl amino butyraldehyde dimethyl acetal

100 gms (0.66 mol) of chloro bromo propane was taken in 100 ml of cyclohexane and 125 gms of 42% caustic soda lye was added at 20-25° C. The mass was stirred for 60 min at 25-30° C. and 100 gms of 40% dimethylamine was added. The mass was stirred for 24 hours at 25-30° C., checked for the absence of chloro bromo propane (limit 5%). 200 ml of cyclohexane was added and organic layer was separated and dried with sodium sulfate. The organic layer was used for next stage without isolation/purification.

25 gms of magnesium turnings and 70 ml of trimethylortho formate was taken in a well-dried flask which was equipped with an addition funnel, thermometer socket and reflux condenser. The reaction mass was heated to 65-70° C. and the above organic layer was added in 4 hours. The reaction mixture was stirred for 2 hours at 65-70° C. and cooled to 25-30° C. The mass was filtered and filtrate was stripped off solvent under reduced pressure. The product was distilled under 10-15 mm/Hg at 145-150° C. to get 28 gms of the >90% pure (GC) 4,4-dimethylamino butyraldehyde dimethyl acetal, which can be used straight away for the next stage

### Example 2

### 4,4-Dimethyl amino butyraldehyde diethyl acetal

100 gms (0.66 mol) of chloro bromo propane was taken in 100 ml of cyclohexane and 125 gms of 42% caustic soda lye was added at 20-25° C. The mass was stirred for 60 min at 25-30° C. and 100 gms of 40% dimethylamine was added. The mass was stirred for 24 hours at 25-30° C. and 200 ml of cyclohexane was added. The organic layer was separated and dried with sodium sulfate. The organic layer was used for next stage without isolation/purification.

20 gms of magnesium turnings was taken the flask and 80 ml of triethylortho formate was taken in a well-dried flask which was equipped with an addition funnel, thermometer socket and reflux condenser. The reaction mass was heated to 65-70° C. and the above organic layer was added in 4 hours. The reaction mixture was stirred for 2 hours at 65-70° C. and cooled to 25-30° C. The mass was filtered and filtrate was stripped off solvent under reduced pressure. The product was distilled under 10 mm/Hg at 135-140° C. to get 30 gms of the >94% pure (GC) 4,4-dimethylamino butyraldehyde dimethyl acetal, which can be used straight away for the next stage

### Example 3

### 3-(2-Dimethylamino)-N-methyl-1H-indole-5-methane sulfonamide succinate

A mixture of 40 gms (159.5 m.mol) of 4-Hydrazino-N-methyl benzene methane sulphonamide, 50 ml of water, 34 gms (211 m.mol) of 4,4-dimethylamino butyraldehyde dimethyl acetal and 80 ml of 2N hydrochloric acid was taken and stirred for 4 hours at 25-30° C. The resulting mixture was basified with sodium carbonate and extracted with chloroform. The chloroform layer and 130 gms of ethyl polyphosphate was stirred at 25-30° C. for 4 hours and then 600 ml of water added. The organic layer was separated and aqueous layer was basified with potassium carbonate and the product was extracted with ethylacetate. The organic layer was distilled off completely under reduced pressure and 40 ml of acetonitrile was added. After 2 hours of cooling at 5° C. the crystals were filtered and dried to give 3.2 gms of crude 3-(2-Dimethylamino)-N-methyl-1H-indole-5-methane sulfonamide. The purity of the product was 82% (HPLC). Melting range of the crude product was 158-162° C.

3.0 gms of the above impure 3-(2-Dimethylamino)-N-methyl-1H-indole-5-methane sulfonamide was taken in 45 ml of acetone and stirred under reflux for 30 min. The acetone solution was treated with 0.5 gm of charcoal and filtered. The carbon bed was washed with acetone and the filtrate was concentrated to 50% of original volume under reduced pressure. The obtained crystals were cooled to 0-5° C. and filtered. Purified base (1.8 gms) has purity of 98.4% (HPLC), melting range 165.5-167.5° C.

1.5 gms of the pure base was taken in 7.5 ml of methanol and 0.95 gms of succinic acid was added at reflux temperature. The mass was stirred under reflux for 30 min, later cooled to 0-5° C. and finally precipitated salt was filtered. The crystals thus obtained were dried at 45-50° C. under vacuum. The product 1.8 gms has the purity of 99.4% (HPLC) and melting range of 166.6-167.8° C.

NMR. (DMSO-d₆),δ includes

2.53 (s, succinate CH₂);

2.74 (s, NHCH₃);

2.89 [s, N(CH)₃];

3.18 [t, j=7.33 Hz, —CH₂N(CH₃)₂]

3.41 [t,—C₂CH₂N(CH₃)₂]

4.54 (s, —CH₂SO₂);

7.25 [d,d j=8.8/1.5 Hz, C₅H];

7.34 (s, C₅H);

7.55 (d, j=8 Hz, C₆H); 7.67 (s, C₃H)

### Example 4

### 3-(2-Dimethylamino)-N-methyl-1H-indole-5-methane sulfonamides succinate

A mixture of 200 gms (0.795 mol) of 4-Hydrazino-N-methyl benzene methane sulphonamide, 500 ml of water, 200 gms (1.058 mol) of 4-dimethylamino butyraldehyde diethyl acetal of example 2 and 400 ml of 2N hydrochloric acid was taken and stirred for 4 hours at 25-30° C. The resulting mixture was basified with sodium carbonate and extracted with chloroform. The chloroform layer and 1.3 kgs of ethyl polyphosphate was stirred at 25-30° C. for 4 hours and then 3 lts of water added. The organic layer was separated and aqueous layer was basified with potassium carbonate and the product was extracted with ethylacetate. The organic layer was distilled off completely under reduced pressure and 200 ml of acetonitrile was added. After 2 hours of cooling at 5° C., the crystals were filtered and dried to give 40 gms of crude 3-(2-Dimethylamino)-N-methyl-1H-indole-5-methane sulfonamide. The purity of the product was 85% (HPLC). Melting range of the crude product was 159-163° C.

25 gms of the above impure 3-(2-Dimethylamino)-N-methyl-1H-indole-5-methane sulfonamide was taken in 500 ml of acetone and stirred under reflux for 30 min. The acetone solution was treated with 2.5 gm of charcoal and filtered. The carbon bed was washed with acetone (25 ml) and the filtrate was concentrated to 50% of original volume under reduced pressure. The obtained crystals were cooled to 0-5° C. and filtered. Purified base (15 gms) has purity of 98.6% (HPLC), melting range 166.6-168.5° C.

10 gms of the pure base was taken in 50 ml of methanol and 7 gms of succinic acid was added at reflux temperature. The mass was stirred under reflux for 30 min, later cooled to 0-5° C. and finally precipitated salt was filtered. The crystals thus obtained were dried at 45-50° C. under vacuum. The product 11.8 gms has the purity of 99.5% (HPLC) and melting range of 166.5-168.0° C.

NMR. (DMSO-d₆),δ includes

2.53 (s, succinate CH₂);

2.74 (s, NHCH₃);

2.89 [s, N(CH)₃];

3.18 [t, j=7.33 Hz, —CH₂N(CH₃)_{2]}

3.41 [t, —CH₂CH₂N(CH₃)₂]

4.54 (s, —CH₂SO₂);

7.25 [d,d j=8.8/1.5 Hz, C₅H];

7.34 (s, C₅H);

7.55 (d, j=8 Hz, C₆H); 7.67 (s, C₃H)

### Example 5

### Preparation of extra pure 3-(2-dimethylamino)-N-methyl-1H-indole-5-methane sulfonamide succinate (through oxalate salt)

A mixture of 3-(2-dimethylamino)-N-methyl-1H-indole-5-methane sulfonamide (25 gms, 98.4% purity) and oxalic acid dihydrate (12 gms) was taken in methanol (100 ml). The mixture was stirred at 45-50° C. for one hour. Then the mixture was cooled to 5-10° C. The precipitated solid was filtered, washed with methanol (20 ml, 5-10° C.), pressed dry and finally dried under vacuum. The melting range was 177.4-178.9° C. Elemental analysis: C: 49.86; H: 5.96, N: 10.90, S: 8.40 (Theoretical C: 49.87; H: 5.97, N: 10.90, S: 8.31)

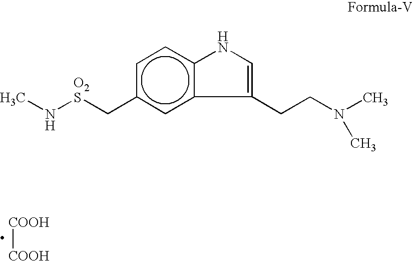

The oxalate salt (32.5 gms) was taken in water (100 ml) and under stirring potassium carbonate (25 gms) was added in installments to obtain a pH of 9.0 to 9.5. The mixture was stirred for another one hour, the pH was checked again and if necessary adjusted to pH to 9.0 to 9.5 by the addition of potassium carbonate. The precipitate was cooled to 10° C., maintained for one hour, filtered, washed with water (25 ml, 5-10° C.) and pressed dry. The dried material (32.5 gms) was taken in acetone (100 ml) and the mixture was stirred under reflux for one hour. Later charcoal (3 gms) was added and the mixture was stirred for further 30 min. The acetone solution was filtered hot and carbon bed was washed with acetone (50 ml, 40-45° C.). The filtrate was concentrated by distilling off half of the acetone, cooled to 0-5° C., maintained for 30 minutes and the precipitated solid was filtered. After washing with cold acetone (25 ml), the precipitate was pressed dry and finally dried under vacuum. Sumatriptan base thus obtained (20 gms) had HPLC purity of 99.4% and melting point of 169-171° C.

20 gms of the pure base was taken in 50 ml of methanol and 14 gms of succinic acid was added at reflux temperature. The mass was stirred under reflux for 30 min, later cooled to 0-5° C. and finally precipitated salt was filtered. The crystals thus obtained were dried at 45-50° C. under vacuum. The product 21.8 gms has the purity of 99.83% (HPLC) and melting range of 167-168.5° C.

NMR. (DMSO-d₆),δ includes

2.53 (s, succinate CH₂);

2.74 (s, NHCH₃);

2.89 [s, N(CH)₃];

3.18 [t, j=7.33 Hz, —CH₂N(CH₃)₂

3.41 [t, —CH₂CH₂N(CH₃)_{2 ]}

4.54 (s, —CH₂SO₂);

7.25 [d,d j=8.8/1.5 Hz, C₃H];

7.34 (s, C₅H);

7.55 (d, j=8 Hz, C₆H); 7.67 (s, C₃H)

### Example 6

### Preparation of extra pure 3-(2-dimethylamino)-N-methyl-1H-indole-5-methane sulfonamide (through citrate salt)

25 gms of crude 3-(2-Dimethylamino)-N-methyl-1H-indole-5-methane sulfonamide (85%) was dissolved in 500 ml of acetone and refluxed for 45 min. Charcoal treatment was given and 50% of solvent was removed under reduced pressure. After cooling the mass to 0-5° C. the obtained crystals were filtered and dried to get the 15 gms of pure 3-(2-dimethylamino)-N-methyl-1H-indole-5-methane sulfonamide with the melting range of the product was 169-170° C. and purity of 98.87% (HPLC)

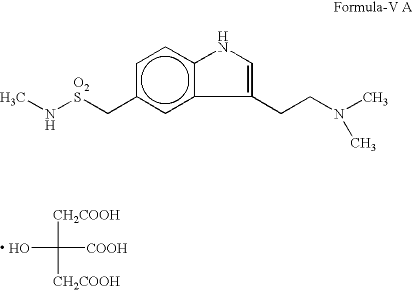

10 gms of pure 3-(2-dimethylamino)-N-methyl-1H-indole-5-methane sulfonamide and 10.0 gms of citric acid was taken in 60 ml of methanol and heated to 35-40° C. After maintaining one hour at 35-40° C., 50% of methanol was removed by distillation under vacuum and then the mass was cooled to 5-10° C.; filtered and washed with 10 ml of chilled methanol. After drying the product under vacuum yielded 16 gms of citrate salt of 3-(2-dimethylamino)-N-methyl-1H-indole-5-methane sulfonamide with melting point of 135-138° C.

Elemental analysis: C: 49.21; H: 5.90, N: 8.59, S: 6.60 (Theoretical C: 49.28; H: 5.95, N: 8.62, S: 6.57)

The citrate salt (10 gms) was taken in water (70 ml) and under stirring potassium carbonate (12 gms) was added in installments to obtain a pH of 9.0 to 9.5. The mixture was stirred for another one hour, the pH was checked again and if necessary adjusted to pH to 9.0 to 9.5 by the addition of potassium carbonate. The precipitate was cooled to 10° C., maintained for one hour, filtered, washed with water (25 ml, 5-10° C.) and pressed dry. The dried material (6.8 gms) was taken in acetone (100 ml) and the mixture was stirred under reflux for one hour. Later charcoal (1 gms) was added and the mixture was stirred for further 30 min. The acetone solution was filtered hot and carbon bed was washed with acetone (50 ml, 40-45° C.). The filtrate was concentrated by distilling off half of the acetone, cooled to 0-5° C., maintained for 30 minutes and the precipitated solid was filtered. After washing with cold acetone (25 ml), the precipitate was pressed dry and finally dried under vacuum. Sumatriptan base thus obtained (5.6 gms) had HPLC purity of 99.4% and melting point of 168.6-170.4° C.

5 gms of the pure base was taken in 25 ml of methanol and 3.5 gms of succinic acid was added at reflux temperature. The mass was stirred under reflux for 30 min, later cooled to 0-5° C. and finally precipitated salt was filtered. The crystals thus obtained were dried at 45-50° C. under vacuum. The product 6.5 gms has the purity of 99.80% (HPLC) and melting range of 167.5-168.9° C.

NMR. (DMSO-d₆),δ includes

2.53 (s, succinate CH);

2.74 (s, NHCH₂);

2.89 [s, N(CH)₃];

3.18 [t, j=7.33 Hz, —CH₂N(CH)₃)₂]

3.41 [t, —CH₂CH₂N(CH)₃)₂]

4.54 (s, —CH₂SO₂);

7.25 [d,d j=8.8/1.5 Hz, C₅H];

7.34 (s, C₅H);

7.55 (d, j=8 Hz, C₆H); 7.67 (s, C₃H)

### Example 7

### Preparation of extra pure 3-(2-dimethylamino)-N-methyl-1H-indole-5-methane sulfonamide succinate (through ascorbate salt)

25 gms of crude 3-(2-Dimethylamino)-N-methyl-1H-indole-5-methane sulfonamide was dissolved in 500 ml of acetone and refluxed for 45 min. Charcoal treatment was given and 50% of solvent was removed under reduced pressure. After cooling the mass to 0-5° C. the obtained crystals were filtered and dried to get the 15 gms of pure 3-(2-ethylamino)-N-methyl-1H-indole-5-methane sulfonamide with the melting range of the product was 165-167 CC and purity of 98.87% (HPLC)

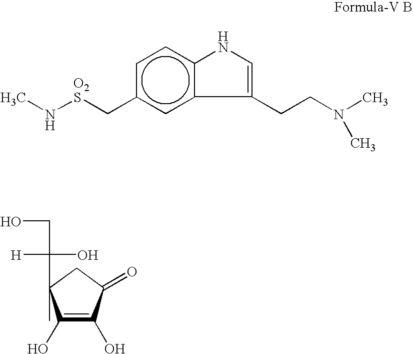

10 gms of pure 3-(2-dimethylamino)-N-methyl-1H-indole-5-methane sulfonamide and 8 gms of ascorbic acid was taken in 50 ml of methanol and heated to 45-50° C. After maintaining one hour at 45-50° C. 25 ml of methanol was distilled, cooled to 5-10° C.; filtered and washed with 10 ml of chilled methanol. After drying the product under vacuum yielded 15 gms of ascorbate salt of 3-(2-dimethylamino)-N-methyl-1H-indole-5-methane sulfonamide with melting point of 115-117° C.

Elemental analysis: C: 50.81; H: 6.02, N: 8.88, S: 6.60 (Theoretical C: 50.95; H: 6.16, N: 8.92, S: 6.79)

The ascorbate salt (10 gms) was taken in water (100 ml) and under stirring potassium carbonate (10 gms) was added in installments to obtain a pH of 9.0 to 9.5. The mixture was stirred for another one hour, the pH was checked again and if necessary adjusted the pH to 9.0 to 9.5 by the addition of potassium carbonate. The precipitate was cooled to 10° C., maintained for one hour, filtered, washed with water (15 ml, 5-10° C.) and pressed dry. The dried material (6.0 gms) was taken in acetone (80 ml) and the mixture was stirred under reflux for one hour. Later charcoal (1 gms) was added and the mixture was stirred for further 30 min. The acetone solution was filtered hot and carbon bed was washed with acetone (50 ml, 40-45° C.). The filtrate was concentrated by distilling off half of the acetone, cooled to 0-5° C., maintained for 30 minutes and the precipitated solid was filtered. After washing with cold acetone (25 ml), the precipitate was pressed dry and finally dried under vacuum. Sumatriptan base thus obtained (5.0 gms) had HPLC purity of 99.4% and melting point of 169-171° C.

5 gms of the pure base was taken in 25 ml of methanol and 3.5 gms of succinic acid was added at reflux temperature. The mass was stirred under reflux for 30 min, later cooled to 0-5° C. and finally precipitated salt was filtered. The crystals thus obtained were dried at 45-50° C. under vacuum. The product 6.4 gms has the purity of 99.81% (HPLC) and melting range of 167-168.9° C.

NMR. (DMSO-d₆),δ includes

2.53 (s, succinate CH₂);

2.74 (s, NHCH₃);

2.89 [s, N(CH)₃];

3.18 [t, j=7.33 Hz, —CH₂(CH₃)₂]

3.41 [t, —CH₂CH₂N(CH₃)₂]

4.54 (s, —CH₂SO₂);

7.25 [d,d j=8.8/1.5 Hz, C₅H];

7.34 (s, C₅H);

7.55 (d, j=8 Hz, C₆H); 7.67 (s, C₃H)

1. A novel process for the preparation of sumatriptan succinate of formula IV of high purity by;
a. isolation of solid sumatriptan base of formula I from a Fisher indole reaction mixture performed between 4-hydrozino-N-methyl benzene methane sulfonamide of formula II and 4-dimethylamino butaraldehyde diethylacetal of formula III,
b. purifying the crude base by crystallization from an organic solvent,
c. converting the crystallized base into an organic salt for further purification,
d. regenerating the base and crystallizing from an organic solvent,
e. converting the base into succinate of extra high purity and better color,

2. A novel process for the preparation of sumatriptan succinate as claimed in claim 1, the isolation of sumatriptan base is achieved in solid form.

3. A novel process for the preparation of sumatriptan succinate as claimed in claim 1(a), wherein the trituration/crystallization is from an organic solvent like acetone or acetonitrile.

4. A novel process for the preparation of sumatriptan succinate as claimed in claim 1(b) wherein the crude base is purified by crystallization from an organic solvent like acetone or acetonitrile.

5. A novel process for the preparation of sumatriptan succinate as claimed in claim 1(c), wherein the purified base is converted into an organic salt like oxalate and or ascorbate for further purification and color.

6. A novel process for the preparation of sumatriptan succinate as claimed in claim 4, wherein the oxalate or ascorbate is formed by treatment of the base of formula I with oxalic acid or ascorbic acid in the mole ratio 1:1-3 in aqueous alkanol at 40° C. to 60° C.

7. A novel process for the preparation of sumatriptan succinate as claimed in claim 1(d), wherein the oxalate or ascarbic acid is converted into highly purified base by treating of the salt in water with alkali or alkaline earth hydroxide, alkali carbonate or bicarbonate ammonia etc.

8. A novel process for the preparation of sumatriptan succinate as claimed in claim 1(d), wherein the purified base is further purified by crystallization from an organic solvent like acetone or acetonitrile.

9. A novel process for the preparation of sumatriptan succinate as claimed in claim 1(e), wherein the highly purified base is converted into a colorless or off-white sample of sumatriptan succinate with HPLC purity of 99.7% to 99.8%.
